(19) United States
(12) Patent Application Publication
(10) Pub. No.: US 2005/0176978 A1
(43) Pub. Date: Aug. 11, 2005

(54) Immobilized iminophosphatranes useful for transesterification

(76) Inventors: John G. Verkade, Ames, IA (US); Victor Shang-Yi Lin, Ames, IA (US); Arunkanti Sarkar, Ames, IA (US)

Correspondence Address: Schwegman, Lundberg, Woessner & Kluth, P.A., P.O. Box 2938, Minneapolis, MN, 55402, US

(21) Appl. No.: 10/989,538

(22) Filed: Nov. 16, 2004

Related U.S. Application Data
(60) Provisional application No. 60/529,550, filed on Dec. 15, 2003.

(51) Int. Cl. C07C 51/43

(52) U.S. Cl. 554/174; 502/162

(57) ABSTRACT

The present invention provides a method for transesterifying an ester, comprising combining the ester, a C₁-C₃ alcohol, and a heterogeneous catalyst of formula (I) or formula (II): 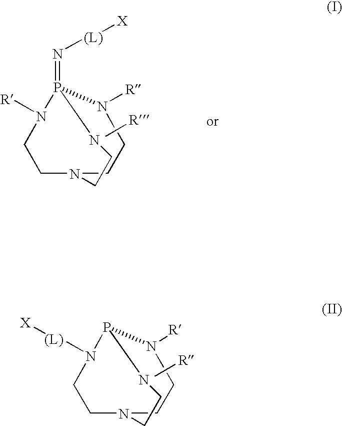
wherein R′, R″ and R′″ are each H, (C₁-C₈)alkyl, (C₆-C₉)aryl, or (alk)₃Si, wherein each alk is (C₁-C₄)alkyl; L is an organic linking moiety and X is a solid support material, and the salts thereof under conditions wherein the catalyst catalyzes the formation of the (C₁-C₃) ester of the acid portion of the ester and the corresponding free alcohol of the ester.

### CROSS-REFERENCE TO RELATED APPLICATIONS

This application claims the benefit under 35 U.S.C. 119 (e) of U.S. Provisional Application No. 60/529,550 filed on Dec. 15, 2003, which is hereby incorporated by reference in its entirety.

### GOVERNMENT FUNDING

This invention was made with Government support of the United States Department of Agriculture Grant No. 2001-52104-11227. The United States Government has certain rights in this invention.

### BACKGROUND OF THE INVENTION

Although the heating value of soybean oil is similar to that of fossil diesel, its use in direct-injection diesel engines is limited by some of its physical properties, especially its viscosity. In general, viscosity of vegetable oil is 10 times greater than that of diesel oil, with consequently poor fuel atomization, incomplete combustion, carbon deposition on the injectors, and fuel build-up in the lubricant oils. The result can therefore be serious engine deterioration which requires the vegetable oils to be modified to diminish their viscosity. Four treatments that have been considered to circumvent the above noted problems are: dilution, microemulsification, pyrolysis and transesterification, among which the latter has been the most commonly employed. The by-product glycerin formed during transesterification is also important because of its numerous applications in the food, cosmetic and pharmaceutical sectors.

Transesterification is an important and fundamental transformation in organic synthesis and the transesterification of soybean oil (SBO) to methyl fatty acid esters (methyl soyate) is currently of particular interest. (Otera, J. Chem. Rev. 1993, 93, 149; Schuchardt, U., J. Braz. Chem. Soc., 1998, 9, 199; Schuchardt, U., J. Mol. Catal. A Chem. 1996, 109, 37; Guo, A., J. Poly. Sci, Part A: Poly. Chem, 2000, 38, 3900; Zagonel, G. F., Preprints of symposia—American Chemical Society, Division of Fuel Chemistry, 2002, 47, 363; Mei, S., Zhongguo Youzhi. 2002, 27, 70; Chang, D. Y. Z., J. Am. Oil. Chem. Soc. 1996, 73, 1549.) SBO contains linoleic (51%), oleic (25%), palmitic (11%), linolenic (9%) and stearic (4%) acids. (Guo, A., J. Poly. Sci, Part A: Poly. Chem, 2000, 38, 3900.)

Methyl soyate (MS), obtained via transesterification of SBO using methanol, is an advantageous alternative to fossil-derived diesel fuel because a) it is easily biodegradable, b) it contains practically no sulfur, c) its transport and storage are not problematic, and, d) it is derived from soybean oil, which sequesters more carbon dioxide from the atmosphere during its production. (Srivastava, A.; Prasad, R. Renewable Sustainable Energy Rev, 2000, 4, 111; Shay, E. G. Biomass Bioenergy 1993, 4, 227; Schwab, A. W., Fuel 1987, 66, 1372; Freedman, B., J. Am. Oil. Chem. Soc. 1986, 63, 1375; Freedman, B., J. Am. Oil. Chem. Soc. 1984, 61, 1638). MS is not only currently useful as a diesel fuel additive, but it is also marketed as an industrial degreasing solvent and as a solvent or diluent for pigments, paints and coatings.

Historically, the transesterification of soybean oil has been carried out using strong bases such as alkali metal hydroxides, carbonates, alkoxides, amines, amidines, guanidines, triamino(imino)phosphoranes and acids such as HCl, H₂SO₄ etc. as catalysts. (Schuchardt, U., J. Braz. Chem. Soc., 1998, 9, 199.) The transesterification reaction using methanol is shown schematically in Scheme 1 below, wherein RCO₂— represents a fatty acid residue, e.g., of (C₈-C₂₂) fatty acids:

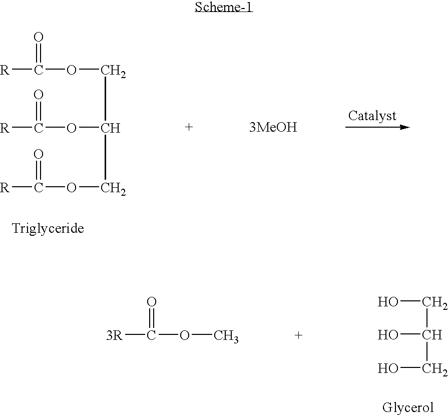

In order to achieve high yields of esters, the alcohol is used in excess. Methanol is inexpensive, possesses a small molecular mass in proportion to the mass of the produced esters, and an advantage of MS is that it gives a higher cetane number than higher esters, such as the ethyl or isopropyl analogs. (Nye, M. J., Southwell, P. H. m Vegetable Oils Diesel Fuel: Seminar III, ARM-NC-28; Bagby, M. O., Pryde, E. H.; Eds.; U.S. Department of Agriculture: Peoria, IL, 1983; p 78; Harrington, K. J.; D'Arcy-Evans, C. Ind. Eng. Chem. Prod. Res. Dev. 1985, 24, 314.) Thus, methanol is the most commonly employed alcohol in the transesterification of vegetable oils. (Chang, D. Y. Z., J. Am. Oil. Chem. Soc. 1996, 73, 1549.)

The transesterification reaction is an equilibrium reaction whose position is accelerated by the presence of a catalyst (typically a strong acid or base). Alkaline alkoxides and hydroxides are considerable more effective catalysts than acid catalysts. (Formo, M. W. J. Am. Oil. Chem. Soc. 1954, 31, 548.) Moreover, acid catalysis is typically conducted at higher temperatures (e.g., 100° C.) as compared with base catalysis. Currently, sodium methoxide (2-10 wt-%) is used as a catalyst in the transesterification of SBO in the presence of methanol. However, the sodium methoxide is converted into sodium chloride by neutralization with hydrochloric acid. The sodium chloride, along with water that is still present in the glycerol, must be removed in processing of the glycerol for its end use. Of the other available catalysts, aluminum isopropoxide, tetraalkoxytitanium compounds, organotin alkoxides and enzymes have been used as homogeneous catalysts. A few heterogeneous catalysts based on guanidines or amines anchored to organic polymers have been reported that operate at the reflux temperature of the solvent employed. Such methods suffer from leaching of organic moieties attached to the support with consequent deterioration of the catalyst sites. The use of homogeneous alkali metal catalysts is not recommended for the transesterification of vegetable oils with high free fatty acid contents because of the relatively large amounts of soaps that are formed, leading to product loss and problems with the separation and purification of the final products.

Phosphatranes and iminophosphatranes include a group of very strong nonionic bases with pKa values for their conjugate acids up to 33 in CH₃CN. See, e.g., P. Kisanger et al., J. Ore. Chem., 65 (2000); J. Verkade, U.S. Pat. Nos. 5,260,436 and 5,051,533. They are useful as catalysts in a variety of organic transformations (J. G. Verkade, Topics in Curr. Chem., 233, 1 (2003)) including transesterification reactions. See, P. Ilankumaran et al., J. Org. Chem., 64, 9063 (1999); P. Ilankumaren et al., J. Org. Chem., 64, 3086 (1999) have reported that using 10 mol-% of catalyst 1a in FIG. 1, methyl benzoate (0.2 M in ethanol) was converted to ethyl benzoate in 84% yield. J. Tang et al., J. Amer. Chem. Soc., 115, 5015 (1993) reported that tris(dialkyl)aminophosphines (1a and 4) react with organic azides (Z-N₃) to give iminophosphoranes in very high yields, e.g., wherein Z is benzyl in FIG. 1. When the aminophosphine is type 1a as shown in FIG. 1, the iminophosphorane obtained (3) is unusually basic compared with its acyclic analogue 5. The strong basicity of 3 is attributed to the stability of protonated 6 incurred through charge delocalization in the latter compound by three nitrogens directly attached to the phosphorus in resonance form 6 and by the fourth nitrogen via transannulation, which enhances the electron density on and basicity of phosphorus in resonance form 7. Although 3 and 5 are potentially useful to transesterify glycerides in vegetable and animal oil feedstocks, they suffer from many of the disadvantages common to homogeneous catalysts, as discussed above.

Therefore a continuous need exists for effective methods to carry out transesterification reactions, so as to efficiently form lower (alkyl) fatty acid esters using triglyceride-containing feedstocks.

### SUMMARY OF THE INVENTION

The present invention provides solid catalysts of the general formula 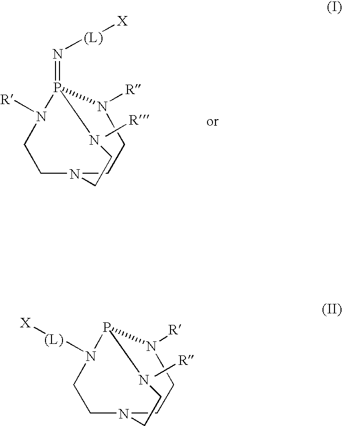
wherein R′, R″ and R′″ are each H, (C₁-C₈)alkyl, preferably (C₁-C₄)alkyl; (C₆-C₉)aryl, or (alk)₃Si, wherein each alk is (C₁-C₄)alkyl; L is an organic linking moiety (“linker”) and X is a solid support material, preferably one that is inert under the reaction conditions used to carry out a transesterification reaction, and the salts thereof.

Preferably, R′, R″ and R′″ are the same group, and X is a solid polymer body. Preferred organic polymers include dendrimers, including highly-branched polymers, as well as conventional polymeric resins based on linear, including cross-linked linear polymers such as polyacrylates, polyalkylenes, polyacrylates, polycarbonates, polymethacrylates, polystyrenes, polysiloxanes, polysilicates, polyacrylamides and the like. Preferred polymers include mesoporous inorganic materials such as silicates, which are discussed in detail below.

Preferred linkers are formed by the reaction of a functionalized substituent Q in the subunit=N-Z or N-Z (See FIGS. 1-5), on the phosphorane precursor of I or II, with a functional group Y on the polymer body, such as a moiety Si—Y on the exterior or interior surface of a mesoporous silicate matrix. Optionally, Q can comprise a tri(C₁-C₄)alkoxysilyl moiety than is bound into the mesoporous silicate matrix during its formation, thus providing catalytic moieties in the pores of the silicate as well as on the surface. Preferred Z moieties thus include vinyl(C₂-C₁₀)alkyl, vinyl(phenyl)(C₂-C₈)alkyl, tri(C₁-C₃)alkoxy(C₂-C₁₀)alkyl and the like. Terminal vinyl groups on Q can react with mercapto-terminated moieties, such as HS(C₂-C₄)alkyl moieties on mesoporous silicates, to yield L groups comprising sulfide bonds. Thus, preferred -L- moieties are (C₂-C₄)alkyl-S—(C₄-C₁₂)alkyl, (C₂-C₁₀)alkylene, alkyaryl, arylalkyl and the like.

Preferably X is a particulate polymer so that the resultant heterogeneous catalyst can be easily removed from the reaction media by filtration or centrifugation, and can be recycled for further use. These immobilized “super base” catalysts deprotonate alcohols such as (C₁-C₃)alkanols to liberate alkoxide ion (the active species in the transesterification catalysis system) in high concentrations. The use of nonionic catalysts of formula (I) or (II) can avoid the side reactions caused by ionic bases and acids, as discussed above.

Intermediates such as 4b-c, 5a-c, 6a-c, 7a-c, 8a-c, 9a-c, 11, 15, 17, 20a-c and 21a-c (FIG. 5) are also within the scope of the invention, and many of them exhibit catalytic activity as well.

The present invention provides a method to prepare a fatty acid (C₁-C₃)alkyl esters from a feedstock, such as a vegetable or an animal oil, comprising one or more fatty acid glycerol esters such as mono-, di- or tri-glycerides and, optionally free fatty acids, comprising combining the feedstocks, a (C₁-C₃) alcohol and a catalyst of formula (I) or (II) under conditions wherein the catalyst catalyzes formation of the corresponding fatty acid (C₁-C₃)alkyl ester of the glycerol esters, glycerol, and optionally the (C₁-C₃)alkyl esters of any free fatty acids that are present in the feedstock. Thus, in one embodiment, the present invention provides a method to transesterify an ester comprising combining the ester, a (C₁-C₃) alcohol and a catalyst of formula (I) or (II) under conditions wherein the catalyst catalyzes the formation of the (C₁-C₃)alkyl ester of the acid portion of the ester and the corresponding free alcohol of the ester. In another embodiment, the present invention provides a method to prepare a lower (alkyl)ester of a fatty acid comprising combining the fatty acid, a lower alkanol and a catalyst of formula I or II under conditions wherein the catalyst catalyzes the formation of the corresponding lower (alkyl)ester. In a preferred embodiment, the fatty acid is present in an organic or synthetic feedstock such as an animal or vegetable oil that comprises a major portion of glycerol fatty acid esters, such as the mono-, di- and/or tri-esters.

Preferably the fatty acid portion of the ester or glyceride is derived from a (C₈-C₂₂) fatty acid, preferably a (C₁₀-C₁₈) fatty acid, which is a saturated alkyl ester that optionally comprises 1-3 CH═CH moieties in the alkyl chain. The (C₁-C₃) alcohols are preferably methanol, ethanol, propanol or i-propyl alcohol, although higher alkanols such as (C₄-C₆) alkanols may be useful in some applications. The alcohol is preferably used in a molar excess over the starting material acid and/or ester component of the feedstock, since such esterification/transesterification reactions are highly reversible.

### BRIEF DESCRIPTION OF THE DRAWINGS

FIG. 1 depicts general synthetic routes to precursors compounds of formula I, (3, and 7), and 5.

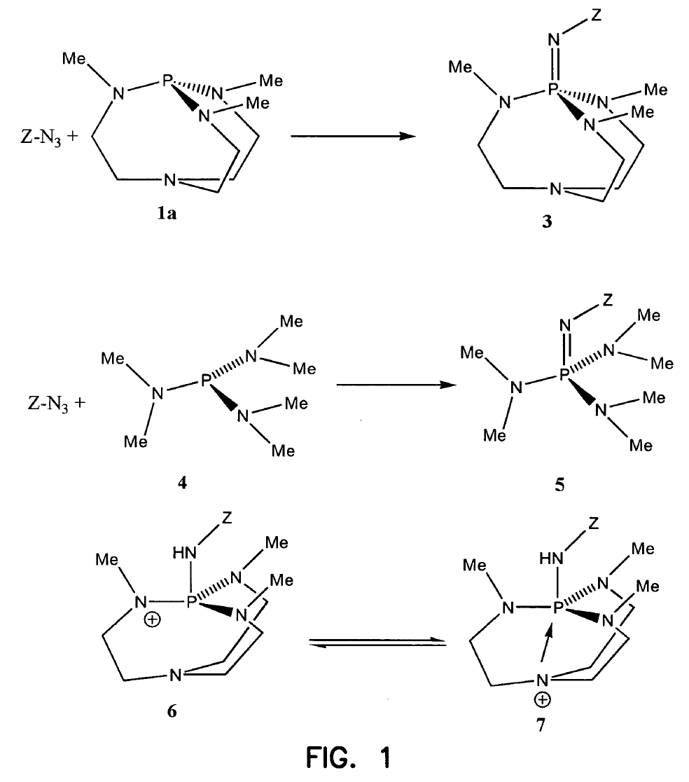

FIG. 2 depicts synthetic routes to specific precursor compounds of formula I, 11 and 13 and 12.

FIG. 3 depicts synthetic routes to specific precursors of compounds of formula I and II (15 and 17).

FIG. 4 depicts a synthetic route to a precursor compound of a compound of formula I (20a) and salt (21a).

FIG. 5 depicts intermediates useful in the present invention.

FIG. 6 is a graph depicting the transesterification of soybean oil to methyl soyate in methanol at 25° C. with mesoporous silica supported superbases.

### DETAILED DESCRIPTION OF THE INVENTION

Mesoporous Silicates

Mesoporous silicates useful in the present method typically have a particle size of about 50 nm to about 1 μm. In one embodiment, the mesoporous silicates have a particle size of at least about 100 nm, or preferably at least about 200 nm. However, particle size is not critical to the practice of the invention and particulate bodies of larger size can be used in some cases, e.g., to facilitate separations. In another embodiment, the mesoporous silicates have a particle size of less than about 750 nm. As conventionally prepared, they are spherical, but they have also been prepared under conditions that yield other shapes such as rods. The articles of the invention can include mesoporous silicates of any shape, provided the pore structure is suitable for receiving the feedstock, e.g., is uniform and of an appropriate diameter.

The mesoporous silicate pores typically have a diameter of from about 1-100 nm. In one embodiment of the invention, the pores have a diameter of at least about 2 nm. In other embodiments, the pores have diameters of greater than about 5 nm, or greater than about 10 nm. Typically, the pores have a diameter of less than about 75 nm or less than about 50 nm.

The mesoporous silicate can be prepared from surfactant micelles of C₁₀-C₁₆ alkyl(trialkyl)ammonium salts in water, followed by introduction into the solution of an alkyl orthosilicate, such as tetraethylorthosilicate (TEOS), and one or more functionalized silanes, such as one or more mercaptoalkyl-, chloroalkyl-, aminoalkyl-, carboxyalkyl-, sulfonylalkyl-, arylalkyl-, alkynyl-, or alkenyl-silanes, wherein the (C₂-C₁₀)alkyl chain is optionally interrupted by —S—S, amido(—C(═O)NR—), —O—, ester(—C(═O)O—), and the like. The aqueous mixture is stirred at moderate temperatures until the silicate precipitates, after which it is collected and dried. The surfactant “template” is then removed from the pores of the ordered silicate matrix, for example, by refluxing the silicate in aqueous-alcoholic HCl. The remaining solvent can be removed from the pores of the silicate by placing it under high vacuum. The functional groups incorporated onto the surface of the pores can be quantified and used as linker moieties to bind to the functionalized super base, or they can be further modified by attaching terminally-functionalized organic linker moieties that can be reacted with functional groups on Z. The polarity of the interior of the pores can also be adjusted by adding other functionalized silanes to the reaction mixture, including ones comprising non-polar inert groups such as aryl, perfluoroalkyl, alkyl, arylakyl and the like. The exterior of the silicate matrix can also be functionalized by grafting organic moieties comprising functional groups thereto. These groups can in turn be employed to link the particles to catalytic moieties such as functionalized “superbase” catalysts.

Superbase-Functionalized Mesoporous Solid Catalysts

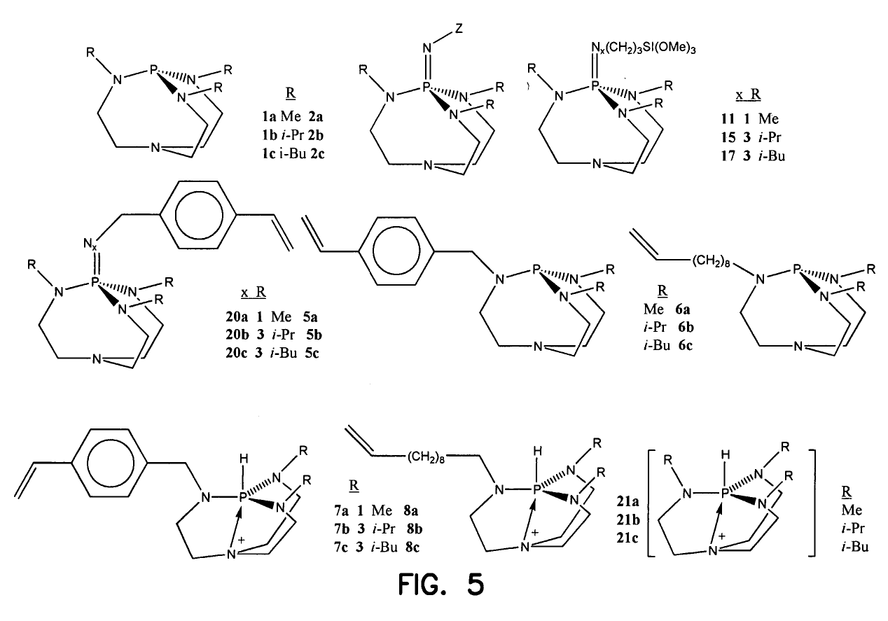

The three commercially available “superbases” of type 1, (1a-1c in FIG. 5) are exceptionally strong nonionic bases with pKa's of ca. 32 in acetonitrile and have superior catalytic activities for a wide variety of reactions including transesterification. See, e.g., U.S. Pat. No. 5,051,533. Their derivatives of type 2a-c are weaker but still very strong bases and catalysts and both types of homogeneous catalysts convert vegetable oils, including soybean oil, to their methyl esters at room temperature. These catalysts deprotonate alcohols to liberate alkoxide ion (the active species in the catalyst system) in concentrations effective for transesterification.

Compounds of types 1 and 2 lend themselves well to chemically bonding to mesoporous silica surfaces via trialkoxysilyl groups (see 11, 15 and 17). The Si(OMe)₃ group linker reacts with up to three SiOH groups in the silica to eliminate MeOH giving robust SiOSi anchors. Precursors 11, 15 and 17 per se homogeneously and efficiently catalyze soybean oil transesterification to soy methyl ester (i.e., 100% conversion of 200 mL of soybean oil in 400 mL of methanol in 24 to 36 h at room temperature using only 0.25 mmol of catalyst). These catalysts are greatly superior to commercially available amine-type nonionic bases or their polymer-mounted forms.

Intermediates 20a, 5c and 6b, have also been prepared, which are designed to take advantage of the vinyl group for linkage to mesoporous solid supports which comprise a mercaptoalkyl functional group already covalently bound thereto. The mercaptoalkyl groups react with vinyl groups to give strong CSC linkages. Such catalysts can liberate even higher concentrations of alkoxide than the first generation systems because of the former's stronger basicity. Synthesis of the other members of groups 20a, 5 and 6 can be accomplished by analogous routes. The presence of more lipophilic P—N nitrogen substituents (i-Pr, i-Bu) in these precursors can facilitate more rapid mass transport of the soy bean oil through the pores of the solid support.

Intermediates 7c and 8b have been prepared as their chloride and triflate salts. By binding members of these classes to mesoporous silica supports containing mercaptopropyl groups, a third generation of catalysts can be prepared by passing sodium hydroxide or methoxide through the catalyst bed, which would replace the chloride or triflate anions with hydroxide or methoxide ions, respectively. Thus, after precursors from these classes have been chemically linked to the mesoporous silica, an anion exchange material is created with pores of sufficient size to pass large molecules such as triglycerides. Compound 21a has been prepared which homogeneously catalyzes complete transesterification of soybean oil at room temperature in a matter of hours. By analogy, the other members of classes 7 and 8 (as chloride or triflate salts) bound to mesoporous supports, followed by hydroxide or methoxide ion exchange will function as catalystic sites on mesoporous supports. Ion exchange regeneration of mesoporous catalysts of all three generations can be accomplished.

Preparation of Superbase Precursors.

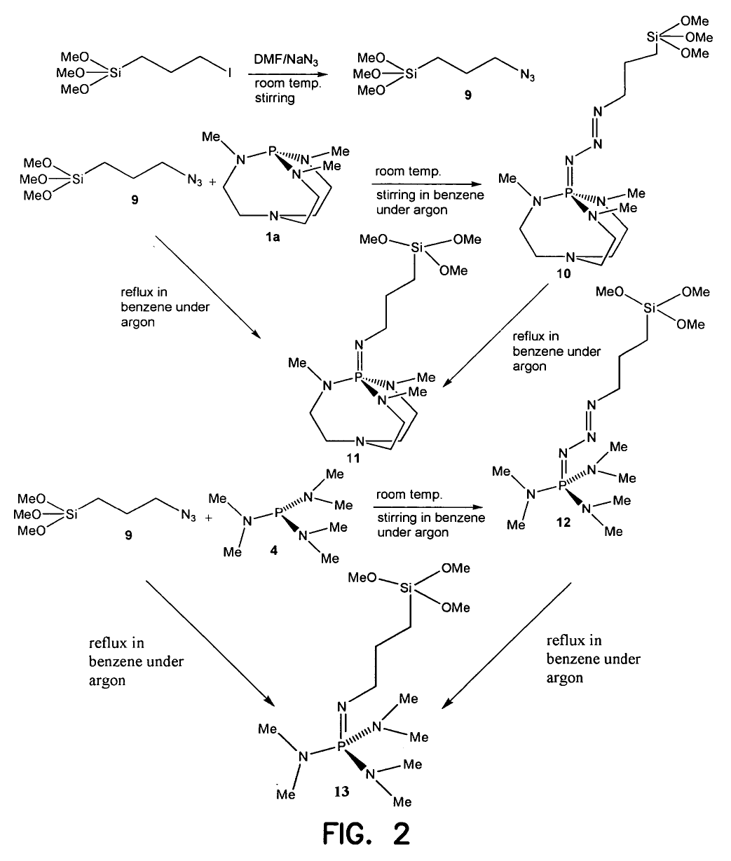

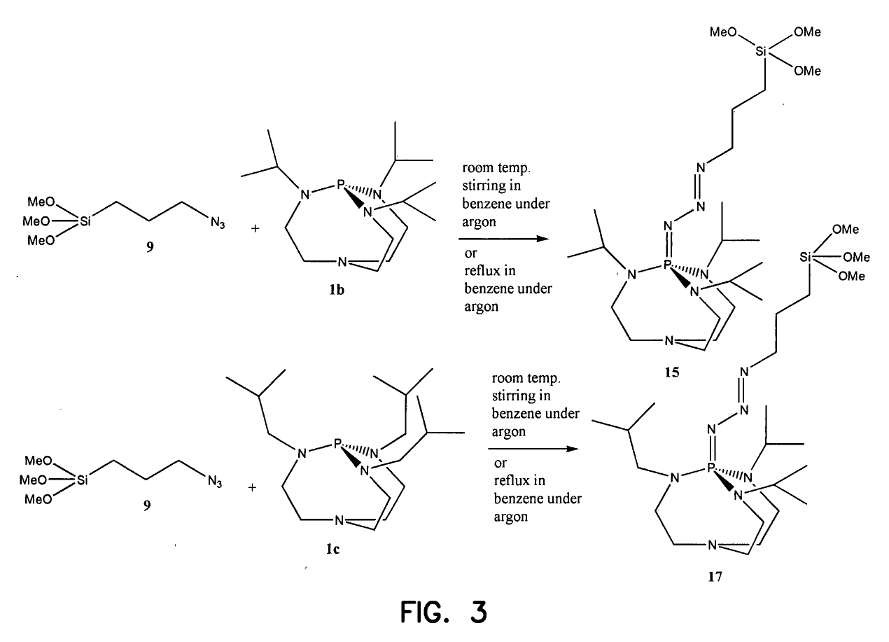

The Staudinger reaction is a two-step process involving the initial electrophilic addition of an alkyl or aryl azide (Z-N₃) to a P(III) center followed by N₂ elimination from the intermediate phosphazide to give the corresponding iminophosphine. In FIG. 2 is shown the reaction of 1-azidopropyl (trimethoxy)silane 9 with compound 1a and 4 in benzene to give azidophosphine compounds 10 and 12, respectively, at room temperature. These products under reflux conditions eliminated nitrogen gas to afford iminophosphines 11 and 13, respectively. Steric hindrance at P(III) does not interfere with the electrophilic addition step of these reactions, but it does suppress decomposition to iminophosphorane, since steric requirements in the four-membered ring transition state are much more rigorous than those in the addition transition state. See Y. Gololobov et al., Tetrahedron, 37 437 (1981); op. cit., 48, 1353 (1992); C. Widauer et al., Eur. J. Inorg. Chem., 1059 (1999). Donor character on the part of P(III) subsituents stabilizes azidophosphines, and this factor apparently also operates in 15 and 17 in FIG. 3. Because of the bulky iso-propyl and iso-butyl groups (which have a greater +I effect than a methyl group) azidophosphines 15 and 17 are very stable and do not eliminate nitrogen to give iminophosphine even under prolonged refluxing in benzene under argon.

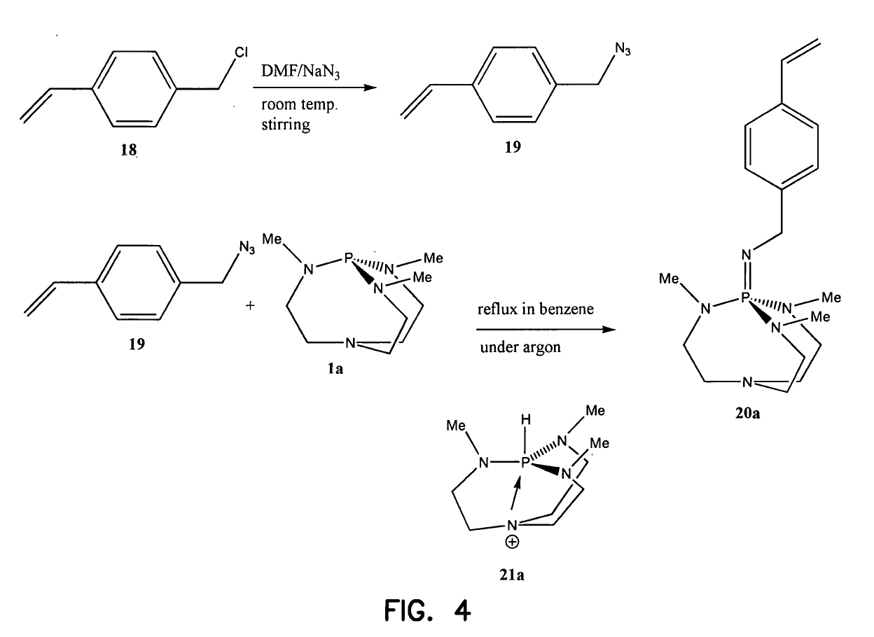

4-Vinyl benzyl azide, prepared from commercially available 4-vinyl benzyl chloride (FIG. 4), reacts with tris(dialkyl)aminophosphine 1a to give iminophosphine 20a, which can be expected to undergo radical polymerization with other monomers, such as acrylates, methacrylates, and styrenes, for example, to give a recyclable polymer-bound iminophosphine catalyst. Iminophosphine 20a can also be reacted with a mesoporous silica-bound alkyl thiol group to give a sulfide linkage that would bind 20a to the mesoporous support, thereby yielding an heterogeneous catalyst system.

Preferably, the esterification/transesterification reaction is carried out at relatively low temperatures, e.g., of about 20-150° C. Although solvent may not be necessary for liquid feedstocks, the reaction can be carried out in the presence of a polar aprotic solvent such as an ether, e.g., THF, dialkylethers, alkoxypolyols, and the like.

As used herein, the term animal oil or vegetable oil includes triglyceride-containing materials from plants (seeds and vegetables), mammals, birds and fish and includes those materials that are solid at room temperature (fats such as lard, tallow, hydrogenated vegetable oils, grease, etc.) as well as materials recognized as oils, such as soybean oil, olive oil, safflower oil, sunflower seed oil, linseed oil, cottonseed oil and the like.

As used herein, the term “alkyl” includes (C₁-C₁₂) alkyl; “lower(alkyl)” includes (C₁-C₃) alkyl.

Thus, in preferred embodiments, the invention provides a method to use the present catalysts to effectively convert a mixture of triglyceride feedstock and a molar excess of a lower alkanol to the corresponding fatty acid (lower)alkyl esters and glycerol. The use of such catalysis provides several advantages over conventional transesterification/esterification: 1. The ability to convert fatty acids into esters in alcohol containing solutions, so that the free fatty acid-containing oils, animal fats, and restaurant deep-fry oils can be used as feedstocks for biodiesel production. 2. The catalysts are solids that function as heterogeneous catalysts that can be separated from the reaction mixture and recycled. 3. The catalysts have high surface areas. 4. Using methanol as the lower alkanol, the catalyst rapidly and under mild conditions converts soybean oil to soybean oil methyl ester plus glycerol (which are easily mechanically separated). The methyl ester (biodiesel) is a viable biodegradable alternative to petroleum-based fuels. Glycerol has a variety of cosmetic and food uses, but it is also under investigation as a biodegradable alternative to petroleum-based ethylene glycol and propylene glycol in aviation de-icing formulations.

### EXAMPLES

### Example 1

Preparation of 1-azidopropyl trimethoxy silane (9): 1-Iodo propyl trimethoxy silane (2.90 g, 10.0 mmol) was added to a heterogeneous solution of NaN₃ (1.48 g, 20.0 mmol) in DMF (10 mL) under argon in a Schlenk flask. The mixture was stirred for 12 h at room temperature. Dry pentane was added to the reaction mixture which was allowed to stir for 3 h and then permitted to settle. The upper pentane layer was carefully cannulated to another Schenk flask under argon. Removal of pentane under vacuum gave (1.85 g) of 1-azidopropyl trimethoxy silane (90% yield). ¹H NMR (400 MHz, CDCl₃): δ 0.64 (t, 2H, SiCH₂), 1.66 (m, 2H, CCH₂C), 3.22 (t, 2H, CH₂N₃), 3.53 (s, 9H, OCH₃). ¹³C NMR (100.5 MHz, CDCl₃): δ 6.44 (SiCH₂), 22.57 (CCH₂C), 50.65 (OCH₃), 53.83(CH₂N₃).

### Example 2

Preparation of azidophosphine (10): To a solution of compound 2 (0.432 g, 2.00 mmol) in dry benzene (15 mL) in a Schlenk flask under argon was added 1-azidopropyl trimethoxy silane (0.410 g, 2.00 mmol) by syringe. The reaction mixture was allowed to stir for 8 h at room temperature. Then removal of benzene under reduced pressure gave of compound 10 in quantitative yield.

### EXAMPLE 3

Preparation of iminophosphorane (11): To a solution of compound 2 (0.432 g, 2.00 mmol) in dry benzene (15 mL) in a Schlenk flask under argon was added 1-azidopropyl trimethoxy silane 9 (0.410 g, 2.00 mmol) by syringe. The reaction mixture was then refluxed under argon for 12 h. After removal of benzene under reduced pressure, 11 was obtained in quantitative yield. ¹H NMR (300 MHz, C₆D₆): δ 1.04 (m, 2H, CH₂Si), 2.02 (m, 2H, CCH₂C), 2.35 (m, 2H, CCH₂), 2.44 (m, 2H, CCH₂), 2.58 (dd, 9H, CH₃), 3.36 (m, 2H, NCH₂), 3.50 (s, 9H, OCH₃). ³¹P NMR (C₆D₆): δ 19.29.

Compound (13). ¹H NMR (300 MHz, C₆D₆): δ 1.05 (m, 2H, CH₂Si), 1.98 (m, 2H, CCH₂C), 2.45 (dd, 18H, CH₃), 3.30 (m, 2H, NCH₂), 3.50 (s, 9H, OCH₃). ⁻P NMR (C₆D₆): δ 25.41.

### Example 4

Preparation of 4-vinyl benzyl azide (19): Commercially available 4-vinyl benzyl chloride (1%) (1.53 g, 10.0 mmol) was added to a heterogeneous solution of NaN₃ (1.48 g, 20.0 mmol) in dry DMF (10 mL) under argon in a Schlenk flask. The mixture was stirred for 12 h at room temperature, extracted with a large excess of ether (500 mL), washed with water (5×20 mL) and dried with Na₂SO₄. Removal of ether at reduced pressure gave 4-vinyl benzyl azide in 84% yield. ¹H NMR (CDCl₃, 300 MHz): δ 7.41 (d, 2H, J=8.10 Hz, Ar—H), 7.25 (d, 2H, J=8.10 Hz, Ar—H), 6.71 (dd, 1H, J=10.70, 17.80, CH═C) 5.75 (d, 1H, J=17.80, C═CH₂), 5.25 (d, 1H, J=10.70, C═CH₂), 4.29 (s, 2H, CH₂N₃). ¹³C NMR (CDCl₃, 75.5 MHz): δ 138.3 136.9, 135.4, 129.1, 127.3, 115.1, 55.2.

### Example 5

Preparation of iminophosphorane (20a): To a solution of compound 1a (0.432 g, 2.00 mmol) in dry benzene (15 mL) in a Schlenk flask under argon was added 4-vinyl benzyl azide (19) (0.318 g, 2.00 mmol) by syringe. The reaction mixture was allowed to reflux for 12 h after which benzene was removed under reduced pressure to give 20a in quantitative yield.

### Example 6

Typical procedure for the tranesterification of soybean oil with catalysts in Table 1: To a solution of soybean oil (200 mL) in methanol (400 mL), 0.25 mmol of catalyst was added. The reaction mixture was stirred at room temperature until the reaction was complete as indicated by the disappearance of the two liquid layers observed at the start of the reaction. Methanol was evaporated under vacuum to give two layers again. This time the layers were methyl soyate (upper layer) and glycerol (lower layer). ¹H NMR spectroscopy indicated that only methyl soyate was present in the upper layer.

### Example 7

Synthesis of Mesoporous Silica Support: The synthesis of SBA-15 mesoporous silica material was accomplished following Q. Huo et al., Nature, 368, 317 (1994). In a typical preparation, a triblock copolymer, Pluronic® 123 (4 g, Aldrich), was dissolved in a solution of 12.1 M aqueous HCl (20 mL of hydrochloric acid in 120 mL of water). After complete dissolution, tetraethoxyorthosilicate (8.2 g, Aldrich) was added to the polymer template solution. The reaction mixture was stirred at 35° C. for 20 h and then the reaction was quenched by terminating the stirring. The slurry product was allowed to age at 90° C. in the same flask for 2 h. The white solid precipitates were isolated by filtration and the crude SBA-15 silica was washed with copious amounts of ethanol. To remove the polymer template, the air-dried product was re-suspended in ethanol (400 mL EtOH for 1 g of solid SBA-15 product) and stirred for 48 h. The completeness of the template removal was monitored by FT-IR. The structure of the purified SBA-15 sample was characterized by BET N₂ absorpotion/desorption isotherms. The results showed a type-IV isotherm, which is characteristic for mesoporous materials with cylindrical pore morphology. The BJH pore size distribution was calculated to be 8 nm in pore diameter.

### Example 8

Soybean Oil Homogeneous Catalytic Transesterification Precursors and with a Novel Ionic Base.

As part of the present invention, transesterification of soybean oil to methyl soyate was performed using catalysts 11 and 15 (prior to mounting them on mesoporous silica supports) with the novel ionic base 21a and also with commercially available polymer-bound DMAP and polymer-bound guanidine at room temperature.

TABLE 1
Soy Bean Oil Transesterification at Room Temperature with
Methanol in the Presence of Various Solid Catalysts

| Catalyst | Time | Conversion |
|---|---|---|
| 11 | 24 h | 100% |
| 15 | 36 h | 100% |
| 21 | 24 h | 100% |
| polymer-bound DMAP | 7 days | partial |
| polymer-bound guanidine^{a} | 24 h | 100%^{b} |

^{a}The guanidine is (guanidinomethyl)polystyrene: 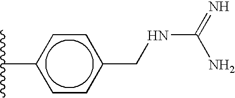
^{b}Recycling took 7 days for 100% conversion.

From the results shown in Table 1, it is clear that 11 and 15 function very efficiently as homogeneous catalysts under very mild conditions. These nonionic catalysts are also superior to the commercially available polymers in the last two entries of this table, because the latter have nonionic bases attached which are weaker than the “superbases”. The ionic base 21 is also very efficient in catalyzing the transesterification of soybean oil. The cation of 21a can be attached via linkers (-L-) to mesoporous silica supports, which will then make it possible to use the OH ion as the catalytically active species, which must remain on the catalyst support to neutralize the positive charge of the cation. Thus, the hydroxide ion is very similar to the ⁻OMe (methoxide) ion in its catalytic properties for transesterification.

### Example 9

Mesoporous Catalyst Preparation: Two SBA-15 type mesoporous catalysts, MA-superbase and TA-superbase, were prepared via the following method with two superbase precursors, namely, 11 and 15, respectively.

A toluene solution of the superbase precursor (2.5 mM) was added to a suspension of the SBA-15 mesoporous silica (1.5 g) in toluene (150 mL). The reaction mixture was refluxed overnight (12 h). The resulting superbase-functionalized mesoporous catalyst was isolated via filtration and washed extensively with toluene. The purified solid catalyst was then lyophilized for 12 h and stored under nitrogen. TGA measurements of the catalysts showed a surface coverage of 6.7×10⁻⁴ mol superbase/gram of catalyst.

### Example 10

Catalytic Activity of the Superbase Mesoporous Catalyst in Converting Soybean Oil to Methyl Soyate (Biodiesel).

In a typical experiment, a mixture of 1 mL of soybean oil and 5 mL of MeOH was charged to a Pyrex flask and stirred at 55° C. Superbase mesoporous catalyst (100 mg) was added to the soybean oil/methanol solution. The extent of transesterification of soybean oil was investigated by solution ¹H NMR spectroscopy. The relevant signals chosen for integration were those of methoxy groups in methyl soyate (3.7 ppm, singlet) and those of the α-methylene protons present in all triglyceride derivatives (2.3 ppm, triplet) of the soybean oil feedstock. The yield of the transesterification was calculated directly from the integrated area (A) of the selected signals using equation (1):
Y %=100*(2A₁/3A₂) (1)
where A1 and A2 are the integrated areas of the methoxy and the methylene protons, respectively (R. Sercheli et al., J. Am. Oil Chem. Soc., 76, 1207 (1999)).

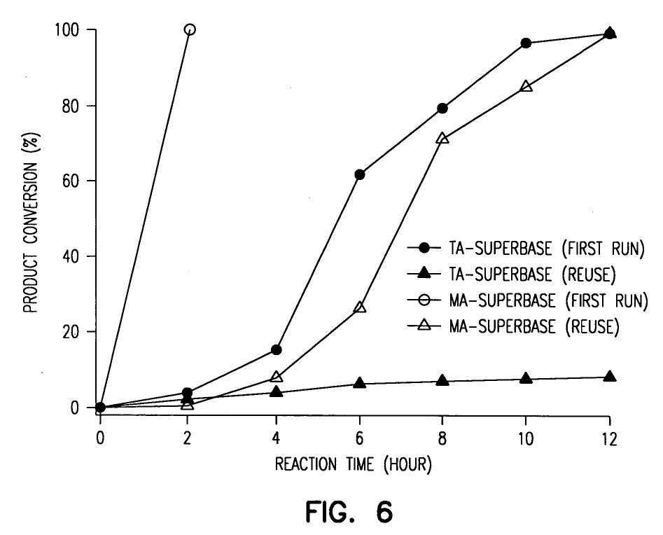

The complete conversion of the soybean oil in methyl soyate was also visualized based on the disappearance of the separated soybean oil phase in the reaction mixture. As shown in FIG. 6, the initial reaction kinetics (first run) of the MA-superbase catalyst containing the attached precursor 11 reached a 100% conversion of the soybean oil to methyl soyate in 20 min. at 25° C. However, the second run of the recycled MA-superbase catalyst showed a slower reaction rate (100% conversion in 12 h), which could be due to two possible reasons: (1) the catalytic functional groups (superbases) might be deactivated (protonated) by the air moisture or they have leached out of the mesopores; (2) the pores might be filled with methyl soyate products (mass-transport problems). Similar behavior was noted for the TA-superbase containing 15 except that is was less active than the MA-superbase in the initial run and also in its re-use.

It should be noted that a mesoporous catalyst made from non-cyclic precursor 13 was only partially effective in transesterifying soybean oil.

All publications, patents and patent applications are incorporated herein by reference. While in the foregoing specification this invention has been described in relation to certain preferred embodiments thereof, and many details have been set forth for purposes of illustration it will be apparent to those skilled in the art that the invention is susceptible to additional embodiments and that certain of the details described herein may be varied considerably without departing from the basic principles of the invention.

1. A method for transesterifying an ester, comprising combining the ester, a C₁-C₃ alcohol, and a heterogeneous catalyst of formula (I) or formula (II): 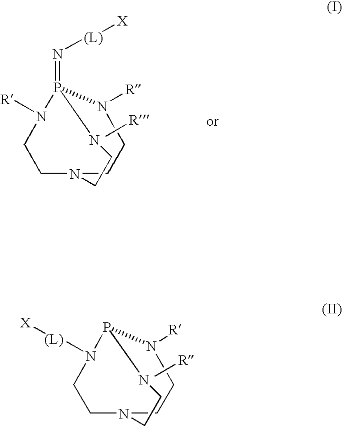
wherein R′, R″ and R′″ are each H, (C₁-C₈)alkyl, (C₆-C₉)aryl, or (alk)₃Si, wherein each alk is (C₁-C₄)alkyl; L is an organic linking moiety and X is a solid support material, and the salts thereof under conditions wherein the catalyst catalyzes the formation of the (C₁-C₃) ester of the acid portion of the ester and the corresponding free alcohol of the ester.

2. A method for preparing fatty acid (C₁-C₃) alkyl esters from a feedstock comprising one or more fatty acid glycerol esters and one or more fatty acids comprising combining the feedstock, a (C₁-C₃) alcohol, and a heterogeneous catalyst of formula (I) or formula (II): 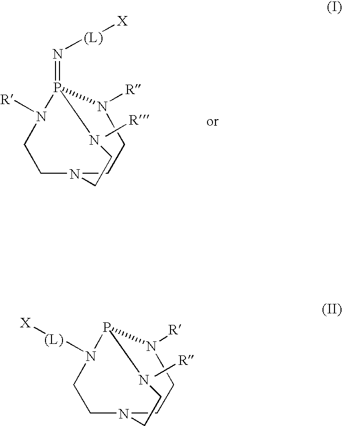
wherein R′, R″ and R′″ are each H, (C₁-C₈)alkyl, (C₆-C₉)aryl, or (alk)₃Si, wherein each alk is (C₁-C₄)alkyl; L is an organic linking moiety and X is a solid support material, and the salts thereof under conditions wherein the mesoporous silicate catalyzes the formation of the corresponding fatty acid (C₁-C₃)alkyl esters.

3. A method for producing a fatty acid (C₁-C₃)alkyl ester, comprising: combining a glyceride-containing vegetable or animal oil, a C₁-C₃ alcohol, and a heterogenous catalyst of formula (I) or formula (II): 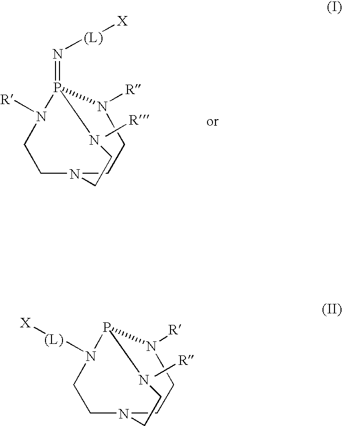
wherein R′, R″ and R′″ are each H, (C₁-C₈)alkyl, (C₆-C₉)aryl, or (alk)₃Si, wherein each alk is (C₁-C₄)alkyl; L is an organic linking moiety and X is a solid support material, and the salts thereof under conditions wherein the catalyst catalyzes formation of the corresponding fatty acid (C₁-C₃)alkyl esters and glycerol.

4. A method for producing methyl soyate, comprising: combining soybean oil, methanol, and a heterogenous catalyst under conditions wherein the mesoporous silicate catalyzes formation of the methyl soyate and glycerol, wherein the catalyst is of formula (I) or (II): 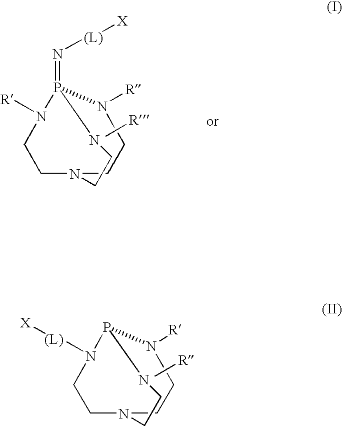
wherein R′, R″ and R′″ are each H, (C₁-C₈)alkyl, (C₆-C₉)aryl, or (alk)₃Si, wherein each alk is (C₁-C₄)alkyl; L is an organic linking moiety and X is a solid support material, and the salts thereof.

5. The method of claim 1, 2, 3 or 4 wherein R′, R″ and R′″ are each (C₁-C₄) alkyl.

6. The method of claim 5 wherein R′, R″ and R′″ are the same group.

7. The method of claim 1, 2, 3 or 4 wherein X is a particulate mesoporous silicate.

8. A solid catalyst of the formula: 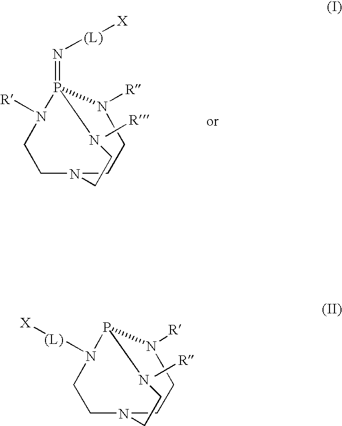
wherein R′, R″ and R′″ are each H, (C₁-C₈)alkyl, (C₆-C₉)aryl, or (alk)₃Si, wherein each alk is (C₁-C₄)alkyl; L is an organic linking moiety and X is a solid support material, and the salts thereof.

9. The catalyst of claim 8 wherein R′, R″ and R′″ are the same group.

10. The catalyst of claim 8 or 9 wherein R′, R″ and R′″ are (C₁-C₄)alkyl.

11. The catalyst of claim 8, 9 or 10 wherein X is a particulate mesoporous inorganic material.

12. The catalyst of claim 11 wherein X is a mesoporous silicate.

13. The catalyst of claim 8, 9 or 10 wherein X is a particulate organic material.

14. The catalyst of claim 13 where X is a linear polymer or a dendrimer.
